(19) United States
(12) Patent Application Publication
(10) Pub. No.: US 2019/0175739 A1
(43) Pub. Date: Jun. 13, 2019

(54) NON-MEDICATED, TEETHING, COOLING COMPOSITION

(71) Applicant: Church & Dwight Co., Inc., Princeton, NJ (US)

(72) Inventors: Jennifer M. Benson, Newtown, PA (US); Albert Yarbrough, Newtown, PA (US); Alysha Moretti, Feasterville, PA (US); Adonis Buelo, Leonardo, NJ (US)

(73) Assignee: Church & Dwight Co., Inc., Princeton, NJ (US)

(21) Appl. No.: 16/212,871

(22) Filed: Dec. 7, 2018

Related U.S. Application Data
(60) Provisional application No. 62/596,395, filed on Dec. 8, 2017.

(51) Int. Cl. A61K 47/46; A61K 47/42; A61K 47/36; A61K 47/38; A61K 47/10; A61K 47/06; A61K 47/24; A61K 9/06; A61K 47/26

(52) U.S. Cl. CPC A61K 47/46; A61K 47/42; A61K 47/36; A61K 47/38; A61K 47/26; A61K 47/06; A61K 47/24; A61K 9/06; A61K 47/10

(57) ABSTRACT

The present disclosure provides compositions suitable for oral use to relieve pain associated with teething in infants and young children. The compositions include a cooling agent in a base formulation that is configured for application to the gums and surrounding tissue and can remain in place for a time sufficient to relive painful sensations, including those arising from teething. The present disclosure further provides methods for relieving pain and/or discomfort in an infant or young child, such as pain and/or discomfort associated with teething.

### CROSS-REFERENCE TO RELATED APPLICATIONS

The present application claims priority to U.S. Provisional Patent Application No. 62/596,395, filed Dec. 8, 2017, the disclosure of which is incorporated herein by reference.

### FIELD OF THE DISCLOSURE

The present disclosure relates to compositions for application to oral tissue, particularly for reducing pain or discomfort. More specifically, the compositions can provide a cooling sensation to reduce discomfort associated with teething in infants and children.

### BACKGROUND

Many infants and young children experience significant discomfort and pain associated with the eruption of teeth through the gums—i.e., “teething.” In the past, compositions for use in relief from such discomfort and pain have included medications, such as benzocaine, and/or homeopathic agents. In 2016, however, the U.S. Food and Drug Administration (FDA) issued a warning stating that homeopathic teething tablets and gel may pose a risk to infants and children. The director of the FDA's Center for Drug Evaluation stated at the time that the FDA has recommended that parents and caregivers not give homeopathic teething tablets and gels to children and they should seek advice from their healthcare professional for safe alternatives. Moreover, compositions containing benzocaine, menthol, and homeopathic agents have been linked to undesirable outcomes in infants, and this has led to restrictions in the use of such materials to children in older age ranges. Accordingly, there is a need in the art for safe alternatives to medicated and/or homeopathic teething tablets and gels, particularly for use in infants and young children.

### SUMMARY OF THE DISCLOSURE

The present disclosure provides compositions that are configured for relieving pain associated with teething. The compositions can exclude ingredients that are contraindicated for use with infants and/or young children (e.g., menthol, homeopathic agents, and medications, such as benzocaine). As such, the compositions can be non-medicated, non-mentholated, and/or non-homeopathic. The compositions specifically can include a cooling agent that is effective to relieve pain by one or more pathways, such as by soothing sore or painful gums and/or by redirecting focus away from the painful sensations and toward the cooling sensations.

In one or more embodiments, a composition for oral administration according to the present disclosure can comprise: a carrier; a humectant; a thickener; a surfactant; and a non-mentholated cooling agent. In further embodiments, the compositions may be further defined in relation to one or more of the following statements, which can be combined in any number or order.

The composition further can comprise an antifoam agent.

The composition further can comprise a sweetener.

The composition further can comprise a soothing agent.

The carrier can be present in an amount of about 25% to about 55% by weight based on the total weight of the composition.

The humectant can be present in an amount of about 5% to about 60% by weight based on the total weight of the composition.

The thickener can be present in an amount of about 0.1% to about 10% by weight based on the total weight of the composition.

The surfactant can be present in an amount of about 0.1% to about 5% by weight based on the total weight of the composition.

The non-mentholated cooling agent can be present in an amount of about 0.1% to about 2% by weight based on the total weight of the composition.

The humectant can comprise one or more polyols.

The thickener can comprise a cellulosic material.

Any one or more of the humectant, the thickener, the surfactant, and the non-mentholated cooling agent can be provided as a plurality of different materials—i.e., a plurality of humectants, a plurality of thickeners, a plurality of surfactants, and/or a plurality of non-mentholated cooling agents.

In one or more embodiments, the present disclosure further can provide methods for reducing discomfort associated with teething in an infant or young child. In particular, the methods can comprise applying a composition as otherwise described herein to oral tissue of the infant or young child. The composition preferably is applied to the gums of the infant or young child. The composition optionally may be applied with massaging of the area of application.

In one or more embodiments, a composition for oral administration according to the present disclosure can comprise: at least one carrier; at least one thickener; and at least one non-mentholated cooling agent. The composition may be further defined in relation to one or more of the following statements, which can be combined in any number or order.

The composition can be substantially in the form of a gel.

The at least one carrier can be aqueous.

The at least one carrier can be present in a total amount of about 40% to about 70% by weight based on the total weight of the composition.

The at least one thickener can be present in a total amount of about 0.1% to about 10% by weight based on the total weight of the composition.

The composition can be substantially in the form of a cream.

The at least one carrier can be non-aqueous.

The at least one carrier can comprise one or both of petrolatum and mineral oil.

The at least one carrier can be present in a total amount of about 40% to about 85% by weight based on the total weight of the composition.

The at least one thickener can be present in a total amount of about 15% to about 50% by weight based on the total weight of the composition.

The at least one thickener can comprise one or both of a gelling agent and a gum.

The composition further can comprise at least one humectant.

The at least one humectant can be present in a total amount of about 5% to about 60% by weight based on the total weight of the composition.

The at least one humectant can comprise one or more polyols.

The composition further can comprise at least one surfactant.

The at least one surfactant present in a total amount of about 0.1% to about 5% by weight based on the total weight of the composition.

The composition further can comprise at least one antifoam agent.

The at least one antifoam agent can be present in a total amount of about 0.01% to about 1% by weight based on the total weight of the composition.

The composition further can comprise at least one sweetener.

The composition further can comprise at least one soothing agent.

The at least one non-mentholated cooling agent can be present in a total amount of about 0.01% to about 2.5% by weight based on the total weight of the composition.

The composition can comprise: about 50% to about 60% by weight of the at least one carrier; about 1% to about 5% by weight of the at least one thickener; about 25% to about 55% by weight of at least one humectant; about 0.5% to about 5% by weight of at least one surfactant; and about 0.01% to about 2.5% by weight of the at least one non-mentholated cooling agent; all of the foregoing weights being based on the total weight of the composition.

The composition can comprise: about 50% to about 85% by weight of the at least one carrier; about 15% to about 50% by weight of the at least one thickener; about 0.01% to about 2.5% by weight of the at least one non-mentholated cooling agent; all of the foregoing weights being based on the total weight of the composition.

In some embodiments, the present disclosure can provide a method of reducing pain or discomfort associated with teething in an infant or young child, the method comprising applying a composition to oral tissue of the infant or young child, wherein the composition comprises: at least one carrier; at least one thickener; and at least one non-mentholated cooling agent. In further embodiments, the method can be defined in relation to one or more of the following statements, which can be combined in any number and order.

The composition used in the method can be substantially in the form of a gel, and the at least one carrier is aqueous.

The composition used in the method can be substantially in the form of a cream, and the at least one carrier is non-aqueous.

The at least one carrier can comprise one or both of petrolatum and mineral oil.

The composition used in the method further can comprise one or more of the following: at least one humectant; at least one surfactant; at least one antifoam agent; at least one sweetener; at least one soothing agent.

The composition used in the method can comprise: about 50% to about 60% by weight of the at least one carrier; about 1% to about 5% by weight of the at least one thickener; about 25% to about 55% by weight of at least one humectant; about 0.5% to about 5% by weight of at least one surfactant; and about 0.01% to about 2.5% by weight of the at least one non-mentholated cooling agent; all of the foregoing weights being based on the total weight of the composition.

The composition used in the method can comprise: about 50% to about 85% by weight of the at least one carrier; about 15% to about 50% by weight of the at least one thickener; about 0.01% to about 2.5% by weight of the at least one non-mentholated cooling agent; all of the foregoing weights being based on the total weight of the composition.

### DETAILED DESCRIPTION OF THE DISCLOSURE

The invention now will be described more fully hereinafter through reference to various embodiments. These embodiments are provided so that this disclosure will be thorough and complete, and will fully convey the scope of the invention to those skilled in the art. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The term “about” used throughout this specification is used to describe and account for relatively small fluctuations. For example, the term “about” can refer to less than or equal to ±5%, such as less than or equal to ±2%, less than or equal to ±1%, less than or equal to ±0.5%, less than or equal to ±0.2%, less than or equal to ±0.1% or less than or equal to ±0.05%. All numeric values herein are modified by the term “about,” whether or not explicitly indicated. A value modified by the term “about” of course includes the specific value.

The present disclosure relates to compositions for oral administration. The compositions can be applied to the gums of infants and young children (e.g., less than 36 months, less than 30 months, less than 24 months, less than 18 months, or less than 12 months of age) to relieve pain or discomfort in the oral cavity, particularly pain or discomfort associated with teething. The compositions particularly can be designed for children that are at least 3 months old.

The present compositions comprise a base formulation that is configured to remain on the site of application for a duration of time sufficient to provide and/or stimulate the desired effect. Preferably, the composition is configured so that the desired effect can persist for a time even after dissolution of the composition in saliva and/or after the composition has been otherwise removed from the area (e.g., by dislocation with the tongue or other oral movement). The compositions are safe for use in the noted population and can be re-applied as necessary to continue with the relieving effect provided by the compositions. The compositions thus are configured to be water soluble yet exhibit a sufficient thickness or other properties so as to allow the composition to remain on or around the site of application for at least a minimum amount of time to allow the transfer and/or release of one or more agents described herein to the noted tissue. The base formulation preferably comprises at least one material from the following groups: a carrier; a humectant; a viscosifying agent; and a surfactant.

In some embodiments, the carrier can be an aqueous material. The aqueous carrier can be any aqueous material safe for consumption. Specifically, the aqueous carrier can comprise water, particularly purified or distilled water. The aqueous carrier may be a buffer solution and/or comprise one or more salts as desired. In further embodiments, the carrier may be a consumable polymeric carrier. For example, polyethylene oxide-based carriers (e.g., polyethylene glycol, or PEG) may be utilized instead of or in combination with an aqueous carrier.

In some embodiments, the carrier can be a non-aqueous material (e.g., a hydrophobic material) and particularly can be a hydrocarbon-containing material. Specifically, a non-aqueous carrier can be a semi-solid or liquid material comprising a mixture of higher alkanes (e.g., C₅ or greater, C₁₀ or greater, or C₁₅ or greater). In particular, mineral oil and/or petrolatum may be utilized as a non-aqueous carrier. As such, in some embodiments, a non-aqueous carrier may comprise a paraffinic material.

The present compositions can comprise one carrier or a combination of carriers. A carrier individually or in a total combined amount can comprise about 25% to about 55%, about 35% to about 55%, or about 40% to about 50% by weight of the total composition based on the overall weight of the composition. As further examples, a carrier individually or in a total combined amount can comprise about 35% to about 75%, about 40% to about 70%, or about 50% to about 60% by weight of the total composition based on the overall weight of the composition. The foregoing ranges may particularly apply in some embodiments to the use of an aqueous carrier and even more particularly to compositions that are substantially in the form of a gel. In further embodiments, it can be useful to include an even greater content of carrier(s). For example, a carrier individually or in a total combined amount can comprise about 50% to about 85%, about 55% to about 80%, or about 60% to about 75% by weight of the total composition based on the overall weight of the composition. Such ranges may particularly apply in some embodiments to the use of a non-aqueous carrier and even more particularly to compositions that are substantially in the form of a cream. Higher carrier ranges likewise may apply to aqueous compositions for use in swabs.

The humectant, in some embodiments, can include one or more materials useful for lowering the viscosity of the composition without limiting the ability of the composition to remain in place on the gums for a time suitable to achieve the desired effect. The humectant can be useful to provide a desired level of “slickness” to the composition so that it provides a soothing feeling on the gums. The humectant further can include one or more materials useful for increasing solubilization of one or more further materials used in the composition. Likewise, the humectant can include one or more materials useful for providing flavoring and/or sweetening to the composition. In some embodiments, the humectant can be a polyol or, more specifically, a sugar alcohol. Non-limiting examples of suitable humectants include glycerin, propylene glycol, xylitol, and sorbitol. In one or more embodiments, a plurality of different humectants may be used. A humectant individually or in a total combined amount can comprise about 5% to about 60% by weight or about 25% to about 55% by weight of the total composition based on the overall weight of the composition. Some humectants may be present in a lower concentration, such as about 5% to about 25%, about 5% to about 20%, or about 5% to about 15% by weight of the total composition. Some humectants may be present in a higher concentration, such as about 30% to about 70%, about 30% to about 50%, or about 30% to about 40% by weight of the total composition.

The thickener can be any material suitable for achieving a suitable consistency such that the composition exhibits a self-sustaining volume and is not rapidly removed from the site of administration. In some embodiments, for example, it can be useful to provide the composition substantially in the form of a gel. In other embodiments, for example, it can be useful to provide the composition substantially in the form of a cream. Suitable thickeners can be chosen to be used alone or in combination to achieve the desired consistency. For example, in some embodiments, suitable thickeners can include gelling agents, such as gelatin and pectin; however, it can be desirable to utilize thickeners that provide thickening without solidification. In some embodiments, cellulosic materials in particular can be used as the thickener. For example, carboxymethylcellulose and other cellulose gums in particular can be useful. Other gums, such as xanthan gum, likewise may be used. In one or more embodiments, a plurality of different thickeners may be used. A thickener individually or in a total combined amount can comprise about 0.1% to about 10%, about 0.5% to about 8%, about 1% to about 5%, or about 1.5% to about 4% by weight of the total composition based on the overall weight of the composition. Such concentrations can be particularly useful, for example, in the formation of compositions substantially in the form of a gel. In other embodiments, a greater total content of thickener can be used. For example, a thickener individually or in a total combined amount can comprise about 15% to about 50%, about 20% to about 40%, or about 25% to about 35% by weight of the total composition based on the overall weight of the composition. Such higher concentrations can be useful, for example, in the formation of compositions substantially in the form of a cream. Likewise, such higher concentrations can be useful in the formation of aqueous compositions for delivery in a swab. The choice of thickener then can be determined based upon the desired end product. For example, to form a cream, it can be desirable to utilize one or more thickeners that would be understood to provide the desired cream consistency. As a further example, to form a less viscous gel for use in a swab, it can be desirable to utilize one or more thickeners that would be understood to provide less structure to the end product. For example, while polyols are described as useful humectants, in order to form a gel with reduced viscosity, one or more polyols may be utilized as a thickener.

A surfactant can be any material useful for stabilizing a mixture and improving solubilization of the various ingredients otherwise discussed herein. Non-ionic surfactants specifically may be preferred in some embodiments. In particular, polyoxyethylene, polyoxypropylene block polymers known as poloxamers can be useful. As an example, poloxamers available under the tradename Pluracare® can be useful. Surfactants such as poloxamers can be particularly useful as a rheology modifier to reduce flowability of the composition without significantly increasing the viscosity of the composition and thus improve contact time of the composition in the mouth. In one or more embodiments, a plurality of different surfactants may be used. A surfactant individually or in a total combined amount can comprise about 0.1% to about 5%, about 0.25% to about 4%, about 0.5% to about 3.5%, or about 1% to about 3% by weight of the total composition based on the overall weight of the composition.

In addition to the foregoing, the composition may include one or more pH adjusting agents. In particular, the pH adjusting agent may be an acidifying agent. For example, organic acids, such as citric acid, may be used. A pH adjusting agent individually or in a total combined amount can comprise about 0.01% to about 2%, about 0.015% to about 1%, about 0.02% to about 0.5%, or about 0.025% to about 0.04% by weight of the total composition based on the overall weight of the composition.

In some embodiments, the composition can include one or more antifoam agents. Any material suitable for reducing or preventing foaming of the composition during mixing may be used. For example, simethicone may be used. An antifoam agent individually or in a total combined amount can comprise about 0.01% to about 1%, about 0.05% to about 0.5%, or about 0.1% to about 0.4% by weight of the total composition based on the overall weight of the composition.

In further embodiments, the composition can include one or more preservatives. Any material suitable for maintaining freshness of the composition can be utilized. For example, potassium sorbate can be used. A preservative individually or in a total combined amount can comprise about 0.01% to about 1%, about 0.05% to about 0.5%, or about 0.1% to about 0.4% by weight of the total composition based on the overall weight of the composition.

In addition to the base formulation, the present composition can include one or more flavoring agents. The flavoring agents can be configured to provide sweetness and/or another desired flavor to the composition. For example, suitable flavoring agents may provide fruit flavors (e.g., strawberry, cherry, banana, apple, pear) to the composition. Non-sugar sweeteners in particular may be included as flavors. For example, sodium saccharin may be used. Similarly, one or more artificial or natural fruit flavors may be used.

In one or more embodiments, the composition can include one or more cooling agents. A cooling agent may be provided singularly in the composition, or a plurality of cooling agents may be used. Further, the cooling agent may be included in combination with one or more flavors. In some embodiments, the flavors used in the composition in particular will include at least one cooling agent.

Although menthol is a known cooling agent, the present compositions particularly can exclude menthol. As such, the compositions can particularly include a non-mentholated cooling agent (i.e., a cooling agent that does not include menthol). Rather, other cooling agents that are safe for consumption by the above-noted population will be utilized. Non-limiting examples of sensorial agents effective for imparting a cooling sensation when contacted with human tissue include the following: [N-(4-cyanomethylphenyl) p-menthanecarboxamide]; 2-isopropyl-5-methylcyclohexyl-4-(dimethylamino)-oxobutanoate; monomenthyl succinate (MMS); menthol ethylene glycol carbonate (MGC); menthol propylene glycol carbonate (MPC); menthone glycerol ketal; (−)-menthyl lactate; 3-(1-menthoxy) propane-1,2-diol (MPD); Coolact® agent 10, or TK-10; 3-(1-menthoxy)-2-methylpropane-1,2-diol; 3-(1-menthoxy)ethanol (Coolact® 5); 3-(1-menthoxy)propan-1-ol; 3-(1-menthoxy)butan-1-ol; (−)-isopulegol (Coolact® P); p-menthane-3,8-diol (Coolact® 38D, PMD38); (−)-monomenthyl glutarate (physcool 2, MMG); N-ethyl-p-menthane-3-carboxamide (WS3); Ethyl 3-(p-menthane-3-carboxamido)acetate (WS5); (1R,2S,5R)—N-(4-Methoxyphenyl)-p-menthanecarboxamide (WS12); 2-isopropyl-N,2,3-trimethylbutyramide (WS23); [N-([ethoxycarbonyl]methyl)-p-menthane-3-carboxamide] (WS14); N,N-dimethyl menthyl succinamide; N-benzo[1,3] dioxol-5-yl-3-p-menthanecarboxamide; N-benzooxazol-4-yl-3-p-menthanecarboxamide; Coolact® 20; and cis & trans p-Menthane-3,8-diol (PM38D, Coolact® 38D); menthyl lactate; camphor; peppermint oil; spearmint oil; eucalyptus oil; and menthyl salicylate. Any of the foregoing materials may be utilized as a non-mentholated cooling agent as discussed herein. Likewise, any of the foregoing materials may be expressly excluded from a non-mentholated cooling agent as discussed herein.

In particular embodiments, a flavor agent may be a specific mixture of flavoring agent(s) and cooling agent(s). For example, a flavor agent may include one or more natural or artificial fruit flavors and also include one or more cooling agents. As a non-limiting example, the flavorant available from Takasago International Corporation under the tradename Intensates® N&A Strawberry Banana Cool Flavor (TAK-171600) combines a cooling agent, a banana flavor, and a strawberry flavor into a single flavor agent mixture that can be particularly useful according to embodiments of the present disclosure. Thus, a non-mentholated cooling agent as discussed herein may comprise a single cooling agent or a plurality of cooling agents and also one or a combination of flavors and/or sweeteners.

A flavorant (including a non-mentholated cooling agent as discussed herein) individually or in a total combined amount can comprise about 0.01% to about 3%, about 0.05% to about 2.5%, about 0.1% to about 2.0%, or about 0.15% to about 1.5% by weight of the total composition based on the overall weight of the composition. A non-mentholated cooling agent in particular can be present individual or in a total combined amount of about 0.01% to about 2.5%, about 0.1% to about 2%, about 0.2% to about 1.5%, or about 0.5% to about 1.25% by weight based on the total weight of the composition.

In some embodiments, the present compositions further can comprise one or more soothing agents. The soothing agent can be any material that is suitable for providing a relaxing sensation, a calming effect, or the like. For example, chamomile can be particularly useful as a soothing agent to aid in relaxation. A soothing agent individually or in a total combined amount can comprise about 0.01% to about 1%, about 0.02% to about 0.5%, or about 0.03% to about 0.2% by weight of the total composition based on the overall weight of the composition.

While the present composition is particularly useful in relation to providing relief of pain and/or discomfort without the need for medications, the composition can include natural ingredients known to elicit pain relief. For example, in some embodiments, the compositions may include eugenol, clove oil, or similar ingredients that are known to provide pain relief.

The present compositions are useful to relieve pain associated with teething because of a sustained cooling sensation that is provided upon contact with the oral tissue. In one or more embodiments, the compositions can expressly exclude medications that are known pain relievers and/or can expressly exclude any compound that is recognized as being a dedicated pain reliever, the compositions can be effective to relieve pain associated with teething by redirecting the focus of the infant or young child away from the painful sensations associated with teething and toward the cooling sensations provided by the present compositions. The cooling sensations can be perceived as comforting to the infant or young child, can block and/or override the painful sensations arising from teething, and can reduce discomfort and irritability associated with teething. The present compositions thus provide the cooling sensation with a base formulation having a pleasing texture that is configured to remain in place on the gums of the infant or young child for a time sufficient to redirect focus away from the painful sensations and the accompanying discomfort associated with teething.

In some embodiments, the present composition is specifically provided substantially in the form of a gel, of a cream, or a similar, thickened composition. For example, a composition substantially in the form of a gel may include at least an aqueous carrier, such as water, and a thickener. As a further, non-limiting example, a composition substantially in the form of a cream may include at least a non-aqueous carrier, such as petrolatum and/or mineral oil, and a thickener. In some embodiments, the compositions may be included on swabs (e.g., pre-applied to a cotton-tipped applicator). In other embodiments, the composition may be provided in a tablet form that may be configured to substantially transition into a thickened, semi-solid (e.g., a gel or a cream) upon contact with liquids in the oral cavity.

Compositions for use with swab applicators may particularly be formulated with an aqueous carrier in some embodiments. For example, known teething swabs comprises a substantially hollow tube (e.g., plastic or other polymeric material) in which the composition is stored, wherein the substantially hollow tube includes an applicator end (e.g., comprising a cotton or other absorptive material) and an opposing, breakable end. When the breakable end is snapped off, the composition within the substantially hollow tube may flow into the applicator end (e.g., be absorbed into the cotton or other absorptive material) over a time of a few seconds to a few minutes. The composition may then be swabbed or otherwise wiped or dabbed onto the area to be treated.

In some embodiments, the present disclosure can relate to methods for reducing pain or discomfort associated with teething in an infant or young child. For example, such method can comprise applying a composition as described herein to oral tissue of the infant or young child. In particular, the composition applied can comprise: at least one carrier; at least one thickener; and at least one non-mentholated cooling agent.

Efficacy of a method as described herein can be evaluated based upon qualitative evidence such as a change in the demeanor of the infant or young child being treated. Specifically, an infant or young child exhibiting symptoms of pain or discomfort associated with teething may cry, chew on items, or touch the painful area with their hands. The associated pain or discomfort may prevent the infant or young child for normal activities, such as eating, playing, sleeping, and can be identified by a parent or caregiver by the overall demeanor of the infant or young child. Evidence of relief of the discomfort or pain may include cessation of the crying, chewing, and/or touching of the mouth area and can be identified by the parent or caregiver by an improvement in the overall demeanor of the infant or young child.

Preferably, the reduction in pain or discomfort is achieved in a minimum time measured from the time of application of the composition to the oral tissue of the infant or young child. For example, the average time to onset of improvements of the symptoms of the pain or discomfort can be about 15 seconds to about 30 minutes, about 30 seconds to about 15 minutes, about 45 seconds to about 10 minutes, or about 60 seconds to about 5 minutes. In some embodiments, duration of the improvement of the symptoms of the pain or discomfort can range from about 10 minutes to about 8 hours, about 15 minutes to about 6 hours, or about 20 minutes to about 4 hours.

Example embodiments of compositions according to the present disclosure are provided in Table 1 through Table 3, wherein Formulation 1 is substantially in the form of a cream, and Formulations 2 and 3 are substantially in the form of a gel. Formulation 2 is an example embodiment of a gel suitable for direct application to the teeth and gums and is structured to be thicker or more viscous than Formulation 3, which is an example embodiment of a gel suitable for delivery in a swab and is structured to be thinner or less viscous than Formulation 2. Preferred concentrations of the respective components of the formulations are provided for reference.

In each composition, the proprietary flavor was Intensates® N&A Strawberry Banana Cool Flavor obtained from Takasago International Corporation (Rockleigh, N.J.). Thus, the compositions exemplified below include a separate flavor/sweetener as well as a flavor/cooling agent. Since the proprietary flavor is a mixture of flavors and cooling agents, the provided concentrations represent the concentration of the overall material, and it is understood that the individual flavor agents and the individual cooing agents are present as fractions of the overall concentration ranges provided below.

TABLE 1
Formulation 1

| Ingredient | Function | Concentration (wt. %) |
|---|---|---|
| Petrolatum | Carrier | 47-60% |
| Mineral oil | Carrier | 1-12% |
| PEG-8 | Carrier | 2-13% |
| Gelatin | Thickener | 7-19% |
| Pectin | Thickener | 7-19% |
| Cellulose gum | Thickener | 1-12% |
| Sodium saccharin | Flavor/seeetener | 0.1-2% |
| Proprietary flavor | Flavor/Cooling agent | 0.1-2% |

TABLE 2
Formulation 2

| Ingredient | Function | Concentration (wt. %) |
|---|---|---|
| Water | Carrier | 50-60% |
| Cellulose gum | Thickener | 1-5% |
| Sorbitol | Humectant | 20-30% |
| Propylene glycol | Humectant | 4-15% |
| Glycerin | Humectant | 1-10% |
| Poloxamer 407 | Surfactant | 0.5-5% |
| Potassium sorbate | Preservative | 0.01-1% |
| Simethicone | Foam control | 0.01-1% |
| Citic acid | pH adjuster | 0.01-1% |
| Sodium saccharin | Flavor/seeetener | 0.1-2% |
| Proprietary flavor | Flavor/Cooling agent | 0.1-2% |
| Chamomile | Soothing agent | 0-1% |

TABLE 3
Formulation 3

| Ingredient | Function | Concentration (wt. %) |
|---|---|---|
| Water | Carrier | 60-75% |
| Propylene glycol | Humectant | 5-15% |
| Glycerin | Thickener | 15-25% |
| Poloxamer 407 | Surfactant | 0.5-5% |
| Potassium sorbate | Preservative | 0.01-1% |
| Sodium benzoate | Preservative | 0.01-1% |
| Sorbic acid | pH adjuster | 0.01-1% |
| Sodium saccharin | Flavor/seeetener | 0.1-2% |
| Proprietary flavor | Flavor/Cooling agent | 0.1-2% |

A home use test was carried out to confirm efficacy of the disclosed compositions in providing relief to infants exhibiting pain associated with teething. Users (N=25) were instructed to apply the composition shown in Table 2 to the gums of an infant exhibiting symptoms of teething pain and then provide a qualitative evaluation of the efficacy of the composition in relieving the pain associated with teething on a scale of 1 (no efficacy) to 10 (complete apparent relief of the teething pain).

Two-thirds of the parents in the home-use test indicated that the composition rated seven (7) or higher in relation to efficacy in relieving pain associated with teething in their infant. One-third of the parents in the home-use test indicated that the composition rated nine (9) or ten (10) in relation to efficacy in relieving pain associated with teething in their infant. The average time duration of relief of the pain and discomfort associated with teething ranged from about 10 minutes to about 2 hours. Most respondents in the home-use test indicated that the average duration of relief of the pain and discomfort associated with teething was about 30 minutes. In addition, two-thirds of the parents in the home-use test indicated that onset of action was as early as two (2) minutes after application of the composition to the gums of the infant.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A composition for oral administration, the composition comprising:
at least one carrier;
at least one thickener; and
at least one non-mentholated cooling agent.

2. The composition of claim 1, wherein the composition is substantially in the form of a gel.

3. The composition of claim 1, wherein the at least one carrier is aqueous.

4. The composition of claim 3, wherein the at least one carrier is present in a total amount of about 40% to about 70% by weight based on the total weight of the composition.

5. The composition of claim 3, wherein the at least one thickener is present in a total amount of about 0.1% to about 10% by weight based on the total weight of the composition.

6. The composition of claim 1, wherein the composition is substantially in the form of a cream.

7. The composition of claim 1, wherein the at least one carrier is non-aqueous.

8. The composition of claim 7, wherein the at least one carrier comprises one or both of petrolatum and mineral oil.

9. The composition of claim 7, wherein the at least one carrier is present in a total amount of about 40% to about 85% by weight based on the total weight of the composition.

10. The composition of claim 7, wherein the at least one thickener is present in a total amount of about 15% to about 50% by weight based on the total weight of the composition.

11. The composition of claim 1, wherein the at least one thickener comprises one or both of a gelling agent and a gum.

12. The composition of claim 1, further comprising at least one humectant.

13. The composition of claim 12, wherein the at least one humectant is present in a total amount of about 5% to about 60% by weight based on the total weight of the composition.

14. The composition of claim 12, wherein the at least one humectant comprises one or more polyols.

15. The composition of claim 1, further comprising at least one surfactant.

16. The composition of claim 15, wherein the at least one surfactant is present in a total amount of about 0.1% to about 5% by weight based on the total weight of the composition.

17. The composition of claim 1, further comprising at least one antifoam agent.

18. The composition of claim 17, wherein the at least one antifoam agent is present in a total amount of about 0.01% to about 1% by weight based on the total weight of the composition.

19. The composition of claim 1, further comprising at least one sweetener.

20. The composition of claim 1, further comprising at least one soothing agent.

21. The composition of claim 1, wherein the at least one non-mentholated cooling agent is present in a total amount of about 0.01% to about 2.5% by weight based on the total weight of the composition.

22. The composition of claim 1, comprising:
about 40% to about 50% by weight of the at least one carrier;
about 1% to about 5% by weight of the at least one thickener;
about 25% to about 55% by weight of at least one humectant;
about 0.5% to about 5% by weight of at least one surfactant; and
about 0.01% to about 2.5% by weight of the at least one non-mentholated cooling agent;
all of the foregoing weights being based on the total weight of the composition.

23. The composition of claim 1, comprising:
about 50% to about 85% by weight of the at least one carrier;
about 15% to about 50% by weight of the at least one thickener;
about 0.01% to about 2.5% by weight of the at least one non-mentholated cooling agent;
all of the foregoing weights being based on the total weight of the composition.

24. A method of reducing pain or discomfort associated with teething in an infant or young child, the method comprising applying a composition to oral tissue of the infant or young child, wherein the composition comprises:
at least one carrier;
at least one thickener; and
at least one non-mentholated cooling agent.

25. The method of claim 24, wherein the composition is substantially in the form of a gel, and the at least one carrier is aqueous.

26. The method of claim 24, wherein the composition is substantially in the form of a cream, and the at least one carrier is non-aqueous.

27. The method of claim 26, wherein the at least one carrier comprises one or both of petrolatum and mineral oil.

28. The method of claim 24, wherein the composition further comprises one or more of the following:
at least one humectant;
at least one surfactant;
at least one antifoam agent;
at least one sweetener;
at least one soothing agent.

29. The method of claim 24, wherein the composition comprises:
about 50% to about 60% by weight of the at least one carrier;
about 1% to about 5% by weight of the at least one thickener;
about 25% to about 55% by weight of at least one humectant;
about 0.5% to about 5% by weight of at least one surfactant; and
about 0.01% to about 2.5% by weight of the at least one non-mentholated cooling agent;
all of the foregoing weights being based on the total weight of the composition.

30. The method of claim 24, wherein the composition comprises:
about 50% to about 85% by weight of the at least one carrier;
about 15% to about 50% by weight of the at least one thickener;
about 0.01% to about 2.5% by weight of the at least one non-mentholated cooling agent;
all of the foregoing weights being based on the total weight of the composition.
